(19) United States
(12) Patent Application Publication
(10) Pub. No.: US 2014/0323443 A1
(43) Pub. Date: Oct. 30, 2014

(54) TIGECYCLINE FORMULATIONS

(71) Applicant: Fresenius Kabi USA, LLC, Lake Zurich, IL (US)

(72) Inventors: Beena UCHIL, Deerfield, IL (US); Shweta MOWLI, Chicago, IL (US)

(21) Appl. No.: 13/952,379

(22) Filed: Jul. 26, 2013

Related U.S. Application Data
(63) Continuation-in-part of application No. 13/873,470, filed on Apr. 30, 2013, now abandoned.

(51) Int. Cl. A61K 31/65; A61K 31/198

(52) U.S. Cl. CPC A61K 31/65; A61K 31/198
USPC 514/154

(57) ABSTRACT

A tigecycline composition includes tigecycline and arginine, where the composition is a solid. The solid composition may be made by forming a liquid mixture including a solvent, tigecycline and arginine, and lyophilizing the liquid mixture.

This application is a continuation-in-part of copending U.S. patent application Ser. No. 13/873,470, filed Apr. 30, 2013, which is incorporated herein by reference in its entirety.

### BACKGROUND

### Background of the Invention

Glycylcyclines are a class of antibiotics that were developed to address the increase in bacterial resistance to the tetracycline class of antibiotics. Glycylcyclines are synthetic derivatives of the tetracyclines, and can be active against the resistant strains of bacteria originally targeted by the tetracyclines, as well as bacteria that have developed resistance to non-tetracycline antibiotics. The first of the glycylcyclines to be approved in the U.S. for use as an antibiotic was tigecycline. The full name for tigecycline is (4S,4aS,5aR,12aS)-9-[2-(tert-butylamino)acetamido]-4,7-bis(dimethyl-amino)-1,4,4a,5,5a,6,11,12a-octahydro-3,10,12,12a-tetrahydroxy-1,11-dioxo-2-naphthacenecarboxamide, and a representative chemical structure is shown in FIG. 1.

Tigecycline has been approved in the U.S. for treatment of a variety of bacterial infections, including complicated skin and skin structure infections, complicated intra-abdominal infections, and community-acquired bacterial lung infections. An approved treatment regimen for adults includes administration of an initial does of 100 milligrams (mg) tigecycline, followed by administration of a maintenance dose of 50 mg tigecycline every 12 hours, where each administration is performed through intravenous infusion over 30-60 minutes.

As tigecycline has poor oral bioavailability, it typically has been provided to medical personnel as a lyophilized solid, which is then reconstituted before intravenous administration to a patient. In one example, a formulation of tigecycline that is commercially available at present is sold under the TYGACIL trademark. TYGACIL™ for Injection (Pfizer Inc.; New York, N.Y., USA) is currently available as a lyophilized powder containing 53 mg of tigecycline and 106 mg lactose monohydrate, and including hydrochloric acid and/or sodium hydroxide as pH modifiers. TYGACIL™ is reconstituted for administration by combining the lyophilized powder with 5.3 milliliters (mL) of a reconstitution liquid (such as 0.9% sodium chloride), to provide a solution having a tigecycline concentration of 10 milligrams per milliliter (mg/mL). An aliquot of 5 mL of this solution, which contains 50 mg tigecycline, is diluted prior to administration with 100 mL of an infusion liquid to provide a final concentration of 0.5 mg/mL. For an initial dose of 100 mg tigecycline, two vials of TYGACIL™ may be reconstituted in 5.3 mL of a reconstitution liquid each, and then both reconstituted liquids may be added to 100 mL of an infusion liquid, to provide a final concentration of 1.0 mg/mL.

Tigecycline degrades under ambient conditions, and this degradation can occur by two pathways. In one pathway, tigecycline can oxidize, such as by forming a double bond between the position 6 and position 5a ring atoms. This oxidation product may be referred to as the “6-ene” impurity, and is presently believed to be represented by the chemical structure shown in FIG. 2, where the arrow points to the double bond between positions 6 and 5a of the ring system. In another pathway, tigecycline can undergo epimerization to reverse the stereochemistry of the position 4 ring atom, which is bonded to one of the dimethylamino substituents. The resulting epimer is presently believed to be represented by the chemical structure shown in FIG. 3, where the arrow points to the position 4 ring atom. Both of these degradation pathways convert the pharmacologically active tigecycline molecule into a species that is inactive or that has an antimicrobial activity that is substantially lower than that of tigecycline.

One of the challenges in preparing and using formulations of tigecycline is that each of these two degradation pathways is favored under a different pH range. Oxidative degradation is favored under basic conditions, whereas epimerization is favored under acidic conditions. Accordingly, a change to the processing or formulation of tigecycline that minimizes one of the degradation pathways typically enhances the other degradation pathway.

One reported approach to minimizing the overall degradation of tigecycline was to control the temperature and oxygen content of the environment surrounding the tigecycline when preparing a lyophilized formulation. By maintaining an aqueous liquid containing the tigecycline at a temperature of 2-8° C. before lyophilization, sparging the water used to form the liquid with an inert gas such as nitrogen, and blanketing the tigecycline with an inert gas while adding it to the water, the amount of tigecycline converted into degradants prior to lyophilization was maintained below 1% for 24 hours. See U.S. Pat. No. 7,704,168. Disadvantages of this approach include the cost and difficulty of maintaining a low temperature and low oxygen content environment prior to lyophilization, and the need for hospital staff to maintain a low temperature and low oxygen content environment during reconstitution and administration of the tigecycline.

In addition to modifying the procedure for preparing a lyophilized tigecycline formulation, various modifications to the chemical composition of the formulation have been reported to improve the stability of tigecycline. Combining tigecycline with a carbohydrate and either an acid or a buffer resulted in lyophilized formulations in which tigecycline was less susceptible to both oxidative degradation and epimerization than when tigecycline was lyophilized with no excipients. Thus, the TYGACIL™ formulation, which includes tigecycline, lactose monohydrate and a pH modifier, has improved stability relative to lyophilized formulations of tigecycline alone. See U.S. Pat. No. 7,879,828.

In another example, tigecycline was combined with ascorbic acid (vitamin C), an ascorbate salt, threonine or serine instead of with a carbohydrate. Lyophilized formulations containing ascorbic acid or threonine stabilized tigecycline at levels comparable to that of TYGACILT™. See Chinese patent publication CN 102138925 A.

Although these modifications to the processing and/or formulation of tigecycline have improved the stability of tigecycline, lyophilized powders containing tigecycline must still be stored in a controlled environment in order to inhibit degradation of the tigecycline. Current protocols for TYGACIL™ require the lyophilized powder to be stored at temperatures from 20° C. to 25° C. Moreover, reconstituted liquids containing tigecycline also must be maintained in a controlled environment. Current protocols for TYGACIL™ allow for reconstituted and diluted liquids to be maintained at room temperature for 24 hours, or to be refrigerated at temperatures from 2° C. to 8° C. for 48 hours. Thus, hospital staff presently is burdened with the need to prepare tigecycline mixtures close to the time of administration, and to monitor the temperature and/or administration time of the reconstituted mixtures, all in the context of caring for a critically infected patient.

It is desirable to have tigecycline formulations that can be stored as lyophilized solids without the need for control of the surrounding temperature. For example, it is desirable for a lyophilized formulation of tigecycline to be stable at a temperature of 25° C. for more than 2 years and/or to be stable at temperatures above 25° C. for at least 2 years. In another example, it is desirable for a reconstituted formulation of tigecycline to be stable at temperatures of 25° C. for more than 24 hours and/or to be stable at temperatures above 25° C. for at least 24 hours. Preferably such stabilized formulations would be convenient to prepare, store, reconstitute and administer.

### BRIEF SUMMARY OF THE INVENTION

A composition is provided that includes tigecycline and arginine, where the composition is a solid.

A composition is provided that includes from 50 to 60 mg tigecycline, and arginine, where the composition is a solid. The mass ratio of arginine to tigecycline is from 1:0.5 to 1:5. When the composition is combined with 5.3 mL of an aqueous carrier liquid to form a solution, the solution has a pH of from 4 to 8.

A method of stabilizing a tigecycline composition is provided. The method comprises forming a liquid mixture including a solvent, tigecycline and arginine, and lyophilizing the liquid mixture to form a solid composition, thereby stabilizing a tigecycline composition.

### BRIEF DESCRIPTION OF THE DRAWINGS

FIG. 1 depicts a chemical structure of tigecycline.

FIG. 2 depicts a chemical structure of the “6-ene” degradation product of the oxidation of tigecycline.

FIG. 3 depicts a chemical structure of the “epimer” degradation product of the epimerization of tigecycline.

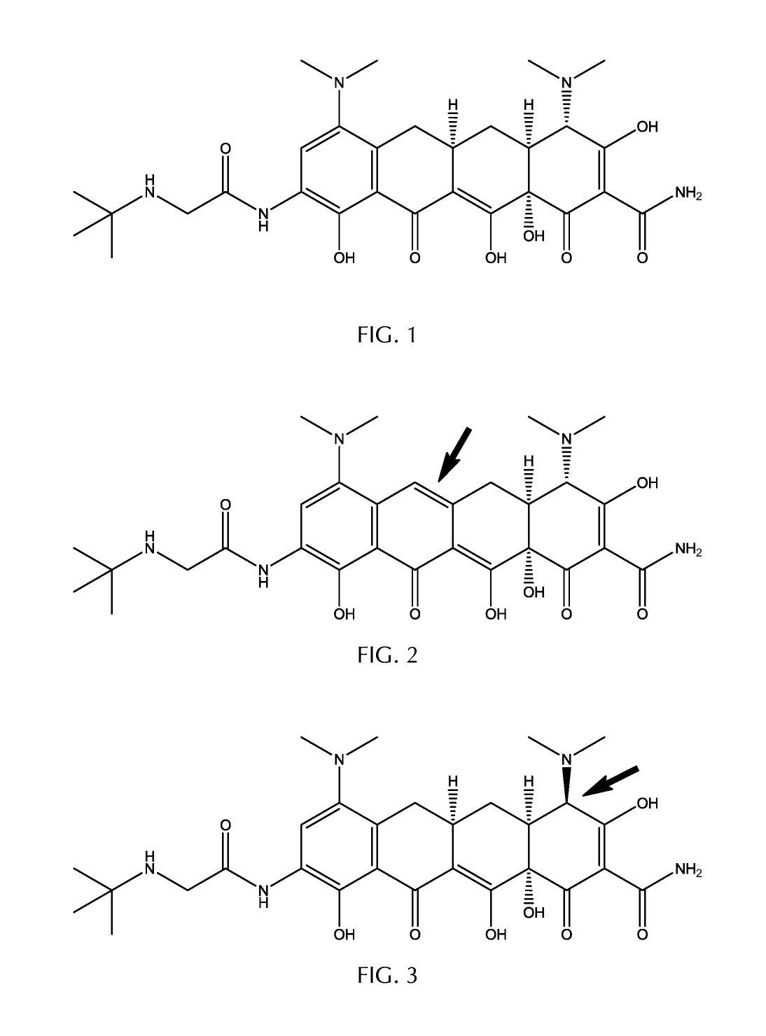

### DETAILED DESCRIPTION OF THE INVENTION

The invention provides a solid composition comprising tigecycline and arginine. The term “solid” means a substance that is not a liquid or a gas. A solid substance may have one of a variety of forms, including a monolithic solid, a powder, a gel or a paste.

As used herein, the term “tigecycline” is intended to include tigecycline in free base form, as well as salts, hydrates, and solvates of tigecycline. Preferably, a tigecycline salt is a pharmaceutically acceptable tigecycline salt, such as tigecycline hydrochloride. The tigecycline can be crystalline or amorphous. If crystalline, the tigecycline can be any polymorphic form.

As used herein, the term “arginine” is intended to include L-arginine, D-arginine, or a mixture of L- and D-arginine, and includes arginine in free base form as well as arginine salts. Preferably, an arginine salt is a pharmaceutically acceptable arginine salt. Exemplary arginine salts suitable for use in the invention include, but are not limited to, hydrochloride, glutamate, butyrate, glycolate, and carbonate salts. In certain embodiments, the arginine is in free base form. Preferably, the arginine is L-arginine in free base form.

A solid composition that includes tigecycline and arginine may include an amount of tigecycline that is sufficient for a single initial dose of tigecycline, or an amount sufficient for a maintenance dose of tigecycline. A solid composition that includes tigecycline and arginine may include an amount of tigecycline that is sufficient for two or more initial doses of tigecycline, or an amount sufficient for two or more maintenance doses of tigecycline. The amount of tigecycline in the composition may be a different therapeutic amount. For example, the amount of tigecycline in the composition may be an amount sufficient for half of an initial dose, or for half of a maintenance dose.

In one example, a solid composition that includes tigecycline and arginine may include from 10 to 200 milligrams (mg) tigecycline. Preferably the composition includes from 25 to 150 mg tigecycline, from 50 to 150 mg tigecycline, or from 50 to 110 mg tigecycline. Presently preferred amounts of tigecycline in the composition include about 53 mg or about 106 mg. The amounts of tigecycline recited herein correspond to the free base forms of tigecycline.

A solid composition that includes tigecycline and arginine may include an amount of arginine sufficient to stabilize the tigecycline. Preferably the amount of arginine in the composition is at most an amount that will dissolve in a sample of aqueous liquid, such as a volume of aqueous liquid used for reconstitution of the solid composition.

In one example, a solid composition that includes tigecycline and arginine may include from 5 to 1,000 mg arginine. Preferably the composition includes from 10 to 750 mg arginine, from 25 to 500 mg arginine, from 50 to 200 mg arginine, or from 50 to 300 mg arginine. In certain preferred embodiments, the composition comprises about 87.5 mg or about 175 mg arginine. The amounts of arginine recited herein correspond to the free base form of arginine.

A solid composition that includes tigecycline and arginine may have a mass ratio of tigecycline to arginine of from 1:0.5 to 1:5. As used herein, the term “mass ratio” of two substances means the mass of one substance (S1) relative to the mass of the other substance (S2), where both masses have identical units, expressed as S1:S2.

In some embodiments, the solid composition has a mass ratio of tigecycline to arginine from 1:0.75 to 1:3, or from 1:1 to 1:2. In other embodiments, the solid composition has a mass ratio of tigecycline to arginine of 1:1, 1:1.25, 1:1.5, 1:1.65, 1:1.75, or 1:2. For a solid composition that contains 53 mg tigecycline, the amount of arginine in the solid composition preferably is from 50 to 100 mg, from 25 to 150 mg, or from 10 to 200 mg.

A solid composition that includes tigecycline and arginine may further include an acid and/or a base. The pH of a saturated solution of tigecycline in water is about 8. The amount of the acid and/or base may be an amount sufficient to provide a pH in the range of from 4 to 6 when a composition containing 53 mg tigecycline is reconstituted in 5.3 mL of an aqueous carrier liquid, such as 0.9% sodium chloride injection. Preferably the amount of the acid and/or base may be an amount sufficient to provide a pH in the range of from 4.25 to 5.75, from 4.5 to 5.75, from 4.5 to 5.5, or from 4.75 to 5.25 when a composition containing 53 mg tigecycline is reconstituted in 5.3 mL of an aqueous carrier liquid. In some embodiments, hydrochloric acid and/or sodium hydroxide is used to adjust pH.

A solid composition that includes tigecycline and arginine may further include one or more other substances. Non-limiting examples of other substances include bulking agents, carriers, diluents, fillers, salts, buffers, stabilizers, solubilizers, preservatives, antioxidants, and tonicity contributors. Substances that may be useful in formulating pharmaceutically acceptable compositions, and methods of forming such compositions, are described for example in Remington: The Science and Practice of Pharmacy, 20th Ed., ed. A. Gennaro, Lippincott Williams & Wilkins, 2000, and in Kibbe, “Handbook of Pharmaceutical Excipients,” 3^{rd} Edition, 2000.

A solid composition comprising tigecycline and arginine may be prepared by any suitable method. In some embodiments, a solid composition comprising tigecycline and arginine is prepared by forming a liquid mixture that includes a solvent, tigecycline, arginine and optionally one or more other substances, and lyophilizing the liquid mixture. The lyophilizing may include freeze-drying the liquid mixture to provide a solid composition. The liquid mixture may include tigecycline and arginine in the amounts described above. The liquid mixture may further include an acid, a base and/or one or more other substances, as described above.

The liquid mixture may include from 0.1 to 10 mL solvent, from 10 to 200 mg tigecycline, and from 5 to 1,000 mg arginine. The liquid mixture may include from 0.5 to 7 mL solvent, from 25 to 150 mg tigecycline, and from 10 to 750 mg arginine. The liquid mixture may include from 0.75 to 5 mL solvent, from 50 to 110 mg tigecycline, and from 25 to 500 mg arginine. The mass ratio of tigecycline to arginine in the liquid mixture may be from 1:0.5 to 1:5, from 1:0.75 to 1:3, or from 1:1 to 1:2.

The solvent, tigecycline, arginine, optional acid, optional base and one or more other optional substances may be combined in any order when forming the liquid mixture. In one example, a liquid mixture may be formed by adding the tigecycline and the arginine to a container including the solvent, and then adding the acid and/or base to achieve the desired pH in the liquid mixture. In another example, a liquid mixture may be formed by combining the arginine and the solvent in a container, adding the acid and/or base to achieve a first desired pH, adding the tigecycline to the container, and adding the acid and/or base to achieve a final desired pH in the liquid mixture. The liquid mixture preferably has a pH of from 4 to 6. In certain preferred embodiments, the pH of the liquid mixture is 5.1.

The liquid mixture including the solvent, tigecycline, arginine, and any other optional ingredients may be lyophilized to form a solid composition, such as by subjecting the liquid mixture to freeze-drying. Freeze-drying of the liquid mixture may include maintaining the liquid mixture in an inert atmosphere, such as nitrogen or argon. Preferably the liquid mixture is placed in glass vials prior to lyophilization, and the amount of the liquid mixture in each vial is based on the amount of tigecycline intended to be present in the final solid composition in the vial.

In a typical lyophilization process, the temperature of the liquid mixture is lowered to a temperature at or below the solidification point of the liquid mixture. If the liquid mixture forms a glass when cooled, the solidification point is the glass transition temperature. If the liquid mixture forms crystals when cooled, the solidification point is the eutectic point. The solidified mixture is then dried under vacuum. Typically, the drying process includes a primary drying step in which the temperature of the solidified mixture is raised gradually while most of the water is removed from the mixture by the vacuum, and a secondary drying step in which the temperature of the solidified mixture is raised further while residual moisture is removed from the mixture by the vacuum. Lyophilization may be complete within 48 hours, or may require additional time. The solid composition resulting from the lyophilization typically is sealed for later use. Details regarding the lyophilization process may be found, for example, in Remington: The Science and Practice of Pharmacy, 20th Ed., ed. A. Gennaro, Lippincott Williams & Wilkins, 2000.

The lyophilized solid composition may be stored for later reconstitution and administration. Preferably the solid composition is stored at a temperature of from 10° C. to 40° C., from 15° C. to 35° C., from 20° C. to 30° C., or about 25° C. Preferably the solid composition is sealed in the glass vial to protect the composition from moisture in the surrounding environment.

A solid composition including tigecycline and arginine may be administered to a patient by combining the composition with an aqueous carrier liquid to form an aqueous mixture, and administering the aqueous mixture into the patient by, for example, injection. Preferably, the aqueous carrier liquid is a pharmaceutically acceptable carrier liquid. Non-limiting examples of pharmaceutically acceptable carrier liquids include water, 0.9% sodium chloride injection, phosphate buffered saline (PBS), 5% dextrose injection, Ringer's solution, and lactated Ringer's solution. The aqueous carrier liquid also may include fixed oils, fatty esters or polyols, particularly if the aqueous mixture for injection is a suspension. The aqueous carrier liquid also may include one or more other substances such as buffers, stabilizers, solubilizers, preservatives and antioxidants. Preferably the solid composition dissolves in the aqueous carrier liquid to form a solution.

In certain embodiments, the aqueous carrier liquid is sodium chloride injection (e.g., solutions containing 0.9%, 0.45%, or 0.225% sodium chloride), sterile water for injection, bacteriostatic water for injection (e.g., which may include, for example, either 0.9% benzyl alcohol or a combination of methylparaben and propylparaben), Ringer's solution, lactated Ringer's solution, or 5% dextrose solution.

The amount of aqueous carrier liquid may be sufficient to provide an initial aqueous mixture containing tigecycline at a concentration of 10 mg/mL. At this concentration, it is convenient to provide a 50 mg or 100 mg dose of tigecycline to a patient, such as by dispensing 5 mL or 10 mL of the mixture into another aqueous liquid, to form a final mixture for administration. While an initial aqueous mixture containing tigecycline at a concentration of 10 mg/mL may be injected into a patient, the presently recommended procedure includes combining the initial mixture with another aqueous liquid to form a final aqueous mixture, which is then administered to a patient.

The amount of aqueous carrier liquid may be sufficient to provide a final aqueous mixture containing tigecycline at a concentration of at most 1 mg/mL. For example, 5 mL of an initial aqueous mixture containing 10 mg/mL tigecycline may be combined with 100 mL of an aqueous carrier liquid to provide a final aqueous mixture containing about 0.5 mg/mL tigecycline (0.476 mg/mL=50 mg/(100 mL+5 mL)). Preferred concentrations of tigecycline in a final aqueous mixture for administration to a patient are from 0.05 to 1.5 mg/mL, from 0.1 to 1 mg/mL, and from 0.3 to 0.7 mg/mL. In certain embodiments, the concentration of tigecycline present in a final aqueous mixture for administration to a patient is about 0.5 mg/mL, e.g., 0.476 mg/mL.

An aqueous mixture formed from the solid composition may be administered to provide an initial dose of 100 mg of tigecycline to a patient. An aqueous mixture formed from the solid composition may be administered to provide a maintenance dose of 50 mg of tigecycline to a patient twice a day. Doses outside of these ranges also may be administered. Typically, an initial dose includes 100 mg tigecycline, and subsequent maintenance doses include 50 mg/mL tigecycline. Higher maintenance doses than 50 mg/mL tigecycline may be advisable under certain conditions, such as an insufficient response by the bacterial infection. Maintenance doses below 50 mg/mL of tigecycline may be advisable under certain conditions, such as for pediatric patients or patients having moderate hepatic impairment.

The invention also provides a method of treating a bacterial infection in a subject. The method comprises combining a solid composition comprising an effective amount of tigecycline and arginine with an aqueous carrier liquid to form a solution, and administering the solution to the subject. In some embodiments, the bacterial infection is a complicated skin or skin structure infection, a complicated intra-abdominal infection, or community-acquired bacterial pneumonia.

Surprisingly, it has been discovered that tigecycline in a solid composition including arginine may be more stable, i.e., less susceptible to degradation, than tigecycline in a solid composition including lactose monohydrate, such as the TYGACIL™ formulation (Pfizer Inc.). As used herein, degradation of tigecycline includes any conversion of tigecycline into a different substance, including but not limited to the 6-ene oxidation product and/or the epimerization product. Degradation of tigecycline also includes any amount of tigecycline impurities that may be derived from a tigecycline synthesis process, such as chemical intermediates of tigecycline, which may be present in a solid composition. Any suitable analytical method can be used to determine of the amount of tigecycline degradation products in a composition. Preferably, the method used to determine the amount of tigecycline degradation products in a composition is high-performance liquid chromatography (HPLC).

In some embodiments, when a solid composition including tigecycline and arginine is stored at 25° C. over a period of 12 months, at most 2.4% of the tigecycline degrades. As used herein, the term “at most x % of the tigecycline degrades” means that following storage for a given length of time, the composition contains x % or less of tigecycline degradation products. Preferably, the amount of tigecycline degradation products present in a composition is calculated based upon the areas under the peaks of tigecycline and the tigecycline degradation products in an HPLC chromatogram.

More preferably, when a solid composition including tigecycline and arginine is stored at 25° C. over a period of 12 months, at most 2%, at most 1.75%, at most 1.5%, at most 1.25%, at most 1%, at most 0.75%, at most 0.5%, or at most 0.3% of the tigecycline degrades. In certain embodiments, when a solid composition including tigecycline and arginine is stored at 25° C. over a period of 12 months the amount of tigecycline degradation is, for example, 0.3%-2.4%, 0.3%-2%, 0.5%-1.5%, 0.5%-1%, or 0.75%-1.25%.

Preferably, when a solid composition including tigecycline and arginine is stored at 25° C. over a period of 12 months, at most 0.5% of the tigecycline is converted to the 6-ene degradant, and at most 1.2% of the tigecycline is converted to the epimer degradant. More preferably, when a solid composition including tigecycline and arginine is stored at 25° C. over a period of 12 months, at most 0.4% of the tigecycline is converted to the 6-ene degradant, and at most 1.0% of the tigecycline is converted to the epimer degradant. More preferably, when a solid composition including tigecycline and arginine is stored at 25° C. over a period of 12 months, at most 0.3% of the tigecycline is converted to the 6-ene degradant, and at most 0.8% of the tigecycline is converted to the epimer degradant. More preferably, when a solid composition including tigecycline and arginine is stored at 25° C. over a period of 12 months, at most 0.2% of the tigecycline is converted to the 6-ene degradant, and at most 0.6% of the tigecycline is converted to the epimer degradant. More preferably, when a solid composition including tigecycline and arginine is stored at 25° C. over a period of 12 months, at most 0.1% of the tigecycline is converted to the 6-ene degradant, and at most 0.5% of the tigecycline is converted to the epimer degradant. More preferably, when a solid composition including tigecycline and arginine is stored at 25° C. over a period of 12 months, at most 0.08% of the tigecycline is converted to the b-ene degradant, and at most 0.4% of the tigecycline is converted to the epimer degradant.

In certain embodiments, solid compositions of the invention that include tigecycline and arginine may tigecycline from degradation for more than 2 years at room temperature (˜25° C.), and for at least 2 years at elevated temperatures above 25° C.

Preferably, when a solid composition including tigecycline and arginine is stored at 40° C. over a period of 6 months, at most 3% of the tigecycline degrades. More preferably, when a solid composition including tigecycline and arginine is stored at 40° C. over a period of 6 months, at most 2.5%, at most 2%, at most 1.5%, at most 1%, at most 0.9%, at most 0.8%, at most 0.7%, at most 0.6%, or at most 0.5% of the tigecycline degrades.

Preferably, when a solid composition including tigecycline and arginine is stored at 40° C. over a period of 6 months, at most 0.5% of the tigecycline is converted to the 6-ene degradant, and at most 2% of the tigecycline is converted to the epimer degradant. More preferably, when a solid composition including tigecycline and arginine is stored at 40° C. over a period of 6 months, at most 0.4% of the tigecycline is converted to the 6-ene degradant, and at most 1.5% of the tigecycline is converted to the epimer degradant. More preferably, when a solid composition including tigecycline and arginine is stored at 40° C. over a period of 6 months, at most 0.3% of the tigecycline is converted to the 6-ene degradant, and at most 1.25% of the tigecycline is converted to the epimer degradant. More preferably, when a solid composition including tigecycline and arginine is stored at 40° C. over a period of 6 months, at most 0.2% of the tigecycline is converted to the 6-ene degradant, and at most 1% of the tigecycline is converted to the epimer degradant. More preferably, when a solid composition including tigecycline and arginine is stored at 40° C. over a period of 6 months, at most 0.1% of the tigecycline is converted to the 6-ene degradant, and at most 0.75% of the tigecycline is converted to the epimer degradant. More preferably, when a solid composition including tigecycline and arginine is stored at 40° C. over a period of 6 months, at most 0.09% of the tigecycline is converted to the 6-ene degradant, and at most 0.65% of the tigecycline is converted to the epimer degradant.

Preferably, when solid compositions containing tigecycline and arginine are stored at 40° C. over a period of 84 days, at most 2%, at most 1.5% or at most 1% of the tigecycline degrades.

Preferably, when solid compositions containing tigecycline and arginine are stored at 55° C. over a period of 2 weeks, at most 2%, at most 1.5% or at most 1% of the tigecycline degrades.

In some embodiments, when a solid composition including tigecycline and arginine is reconstituted with an aqueous carrier liquid to form a solution, the solution may be stable for more than 24 hours at 25° C., and for at least 24 hours at elevated temperatures above 25° C. In certain embodiments, when the solution is stored at 25° C. over a period of 24 hours, at most 2% of the tigecycline degrades. Preferably, when the solution is stored at 25° C. over a period of 24 hours, at most 1.75%, 1.5%, 1.25% or 1% of the tigecycline degrades. In certain embodiments, the solution has a tigecycline concentration of about 10 mg/mL, about 1 mg/mL, or about 0.5 mg/mL.

Preferably, when a solid composition including tigecycline and arginine is reconstituted with an aqueous carrier liquid to form a solution having a tigecycline concentration of about 1 mg/mL, and the solution is stored at 25° C. over a period of 24 hours, at most 0.5% of the tigecycline is converted to the 6-ene degradant, and at most 1% of the tigecycline is converted to the epimer degradant. More preferably, at most 0.4% of the tigecycline is converted to the 6-ene degradant, and at most 0.8% of the tigecycline is converted to the epimer degradant. More preferably, at most 0.3% of the tigecycline is converted to the 6-ene degradant, and at most 0.7% of the tigecycline is converted to the epimer degradant. More preferably, at most 0.2% of the tigecycline is converted to the 6-ene degradant, and at most 0.6% of the tigecycline is converted to the epimer degradant. More preferably, at most 0.1% of the tigecycline is converted to the 6-ene degradant, and at most 0.5% of the tigecycline is converted to the epimer degradant.

The following examples further illustrate the invention but should not be construed as in any way limiting its scope

### Example 1

This example demonstrates the stability of compositions comprising tigecycline and arginine formed by lyophilizing solutions having a range of acidic pHs.

Solid compositions were formed by dissolving 87.5 mg arginine in water and adjusting the solution to an acidic pH. Tigecycline (53 mg) was added to this solution, providing a mass ratio of tigecycline to arginine of 1:1.65. The pH of individual solutions was adjusted to 4.5, 4.75, 5.1, 5.25 or 5.5. The resulting solutions were clear, with no visible particles. The solutions were then filtered and lyophilized to form solid compositions. The lyophilization procedure was adjusted as need for each type of composition, as the collapse temperature of the composition was affected by the amount of arginine and by the pH. The general procedure, however, included reducing the temperature of the lyophilization solutions to −45° C., performing a primary drying at a temperature of from −35 to −15° C. and under a vacuum of from 50-200 milliTorr (mTorr), and performing a secondary drying at a temperature of 25° C. or 40° C. To ensure the lowest initial impurities possible, the solutions for lyophilization were prepared at low temperature, using a nitrogen blanket and purging.

Each lyophilized composition was sealed in a 5 mL vial with a 13 mm rubber stopper, or in a 10 mL vial with a 20 mm rubber stopper and stored at 25° C., 40° C. or 55° C. (all temperatures±2° C.). The stoppers were STELMI 6720GC (American Stelmi Corporation, Princeton, N.J.). After 4, 8 and 12 weeks at 25° C. or 40° C., and after 2 and 4 weeks at 55° C., a portion of the vials were visually inspected with regard to the color of the solid, and were then reconstituted with 5.3 mL of 0.9% sodium chloride injection, USP. The reconstituted liquids were visually inspected, and were then analyzed by HPLC to determine the concentrations of tigecycline and of any impurities, including the 6-ene oxidation product and the epimerization product. Table 1 lists the results of these analyses for lyophilized compositions stored at 25° C., Table 2 lists the results for lyophilized compositions stored at 40° C., and Table 3 lists the results for lyophilized compositions stored at 55° C.

TABLE 1
Stability at 25 ± 2° C. of tigecycline in solid compositions
containing arginine.

| pH at | Time | pH at | Moisture | Impurities (%) | | |
|---|---|---|---|---|---|---|
| lyophilization | (weeks) | reconstitution | (%) | 6-ene | Epimer | Total |
| 4.5 | 0 | 4.350 | 0.3188 | 0.08 | 0.39 | 0.69 |
| | 4 | 4.343 | — | 0.04 | 0.39 | 0.70 |
| | 8 | 4.294 | — | 0.08 | 0.49 | 0.90 |
| | 12 | 4.28 | — | 0.11 | 0.48 | 0.97 |
| 4.75 | 0 | 4.613 | 0.2506 | 0.10 | 0.32 | 0.66 |
| | 4 | 4.612 | — | 0.04 | 0.34 | 0.64 |
| | 8 | 4.294 | — | 0.08 | 0.34 | 0.78 |
| | 12 | 4.54 | — | 0.12 | 0.39 | 0.92 |
| 5.1 | 0 | 4.950 | 0.2503 | 0.10 | 0.24 | 0.60 |
| | 4 | 4.984 | — | 0.06 | 0.28 | 0.64 |
| | 8 | 4.967 | — | 0.10 | 0.29 | 0.75 |
| | 12 | 4.92 | — | 0.13 | 0.32 | 0.85 |
| 5.25 | 0 | 5.091 | 0.3034 | 0.12 | 0.24 | 0.60 |
| | 4 | 5.106 | — | 0.06 | 0.27 | 0.64 |
| | 8 | 5.091 | — | 0.11 | 0.28 | 0.74 |
| | 12 | 5.03 | — | 0.15 | 0.33 | 0.87 |
| 5.5 | 0 | 5.383 | 0.2875 | 0.15 | 0.22 | 0.67 |
| | 4 | 5.368 | — | 0.08 | 0.22 | 0.64 |
| | 8 | 5.361 | — | 0.15 | 0.27 | 0.82 |
| | 12 | 5.30 | — | 0.20 | 0.32 | 0.99 |

TABLE 2
Stability at 40 ± 2° C. of tigecycline in solid compositions
containing arginine.

| pH at | Time | pH at | Moisture | Impurities (%) | | |
|---|---|---|---|---|---|---|
| lyophilization | (weeks) | reconstitution | (%) | 6-ene | Epimer | Total |
| 4.5 | 0 | 4.350 | 0.3188 | 0.08 | 0.39 | 0.69 |
| | 4 | 4.329 | — | 0.04 | 0.53 | 0.84 |
| | 8 | 4.276 | — | 0.09 | 0.62 | 1.05 |
| | 12 | 4.28 | — | 0.13 | 0.69 | 1.21 |
| 4.75 | 0 | 4.613 | 0.2506 | 0.10 | 0.32 | 0.66 |
| | 4 | 4.606 | — | 0.04 | 0.43 | 0.76 |
| | 8 | 4.572 | — | 0.10 | 0.50 | 0.93 |
| | 12 | 4.54 | — | 0.14 | 0.60 | 1.14 |
| 5.1 | 0 | 4.950 | 0.2503 | 0.10 | 0.24 | 0.60 |
| | 4 | 4.986 | — | 0.05 | 0.35 | 0.71 |
| | 8 | 4.973 | — | 0.11 | 0.40 | 0.83 |
| | 12 | 4.93 | — | 0.15 | 0.46 | 0.99 |
| 5.25 | 0 | 5.091 | 0.3034 | 0.12 | 0.24 | 0.60 |
| | 4 | 5.092 | — | 0.06 | 0.33 | 0.70 |
| | 8 | 5.053 | — | 0.12 | 0.43 | 0.89 |
| | 12 | 5.03 | — | 0.19 | 0.53 | 1.16 |
| 5.5 | 0 | 5.383 | 0.2875 | 0.15 | 0.22 | 0.67 |
| | 4 | 5.352 | — | 0.09 | 0.33 | 0.77 |
| | 8 | 5.361 | — | 0.16 | 0.37 | 0.93 |
| | 12 | 5.30 | — | 0.23 | 0.47 | 1.16 |

TABLE 3
Stability at 55 ± 2° C. of tigecycline in solid compositions
containing arginine.

| pH at | Time | pH at | Moisture | Impurities (%) | | |
|---|---|---|---|---|---|---|
| lyophilization | (weeks) | reconstitution | (%) | 6-ene | Epimer | Total |
| 4.5 | 0 | 4.350 | 0.3188 | 0.08 | 0.39 | 0.69 |
| | 2 | — | — | 0.08 | 0.75 | 1.06 |
| | 4 | — | — | 0.04 | 0.87 | 1.19 |
| 4.75 | 0 | 4.613 | 0.2506 | 0.10 | 0.32 | 0.66 |
| | 2 | — | — | 0.09 | 0.65 | 0.99 |
| | 4 | — | — | 0.04 | 0.78 | 1.13 |
| 5.1 | 0 | 4.950 | 0.2503 | 0.10 | 0.24 | 0.60 |
| | 2 | 4.992 | — | 0.10 | 0.53 | 0.90 |
| | 4 | 4.980 | — | 0.06 | 0.68 | 1.07 |
| 5.25 | 0 | 5.091 | 0.3034 | 0.12 | 0.24 | 0.60 |
| | 2 | — | — | 0.11 | 0.54 | 0.94 |
| | 4 | — | — | 0.06 | 0.65 | 1.04 |
| 5.5 | 0 | 5.383 | 0.2875 | 0.15 | 0.22 | 0.67 |
| | 2 | — | — | 0.15 | 0.50 | 0.97 |
| | 4 | — | — | 0.09 | 0.63 | 1.10 |

The pH of the tigecycline solutions prior to lyophilization affected the stability of the tigecycline in the resulting solid compositions. For the temperatures studied, the concentration of the 6-ene oxidation product was lower in compositions formed from solutions having more acidic pH's of 4.5 and 4.75, and was higher in compositions formed from solutions having less acidic pH's of 5.25 and 5.5. In contrast, the concentration of the epimer was higher in compositions formed from solutions having more acidic pH's of 4.5 and 4.75, and was lower in compositions formed from solutions having less acidic pH's of 5.25 and 5.5. The overall concentration of impurities was lowest in the solutions formed from lyophilization compositions having a pH of 5.1 or 5.25.

Other variables besides pH were examined. For example, some samples of the solid compositions were stored in upright vials, whereas other samples were stored in inverted vials in which the powder of the composition was in contact with the rubber stopper. No significant difference in tigecycline stability was observed between the two orientations of the vials.

In another example, the solid compositions were stored in vials sealed with rubber stoppers that had been dried in an oven at 105° C. for 5 hours after sterilization, or were stored in vials sealed with rubber stoppers that were not dried after sterilization. Although the moisture content was slightly lower for solid compositions stored using dried stoppers, no significant difference in tigecycline stability was observed between the two types of stoppers.

The results of this example demonstrated that compositions comprising tigecycline and arginine formed by lyophilizing solutions having a range of acidic pHs are storage stable at 25° C., 40° C., and 55° C.

### Example 2

This example demonstrates that solid compositions comprising tigecycline and arginine are stable in vials of different sizes.

Solid compositions formed from lyophilization compositions having a pH of 5.1 were stored in 10 mL vials sealed with a 20 mm stopper, rather than in 5 mL vials with a 13 mm stopper. Compositions in the 10 mL vials also were analyzed for the effects of stopper drying and/or vial orientation on tigecycline stability. No significant difference in tigecycline stability was observed between the two orientations of vials or between the two types of stoppers. Table 4 lists the results of the analyses for solid compositions stored in 5 mL or in 10 mL vials. The stability trends for compositions in the 10 mL vials were similar to the trends observed for compositions in the 5 mL vials.

TABLE 4
Stability of tigecycline in solid compositions containing
arginine, in 5 mL vials and 10 mL vials.

| Temperature | Time | Vial size | Moisture | Impurities (%) | | |
|---|---|---|---|---|---|---|
| (° C.) | (weeks) | (mL) | (%) | 6-ene | Epimer | Total |
| — | 0 | 5 | 0.2503 | 0.10 | 0.24 | 0.60 |
| — | 0 | 10 | 0.2079 | 0.05 | 0.13 | 0.51 |
| 25 ± 2 | 8 | 5 | — | 0.10 | 0.29 | 0.75 |
| | 8 | 10 | — | 0.06 | 0.21 | 0.66 |
| 40 ± 2 | 4 | 5 | — | 0.05 | 0.35 | 0.71 |
| | 4 | 10 | — | 0.08 | 0.33 | 0.83 |
| | 8 | 5 | — | 0.11 | 0.40 | 0.83 |
| | 8 | 10 | — | 0.09 | 0.42 | 0.91 |
| 55 ± 2 | 2 | 5* | — | 0.10 | 0.52 | 0.90 |
| | 2 | 10* | — | 0.09 | 0.51 | 1.02 |

*Vials were inverted during storage.

The results of this example demonstrated that solid compositions comprising tigecycline and arginine are stable in 5 mL vials and 10 mL vials.

### Example 3

This example demonstrates that solid compositions comprising tigecycline and arginine are more stable than solid compositions comprising tigecycline and an excipient other than arginine.

The stability of solid compositions comprising tigecycline and arginine was compared with stability data reported for solid compositions comprising tigecycline and one of the following excipients: lactose monohydrate, ascorbic acid, threonine or serine. Table 5 lists the results of stability analyses of lyophilized compositions containing tigecycline and one of a variety of excipients. The results for the compositions containing arginine are reproduced from Table 2 above, for the lyophilized solids formed from a liquid having a pH of 5.1. The results for the conventional compositions containing lactose monohydrate are reproduced from U.S. Pat. No. 7,879,828. The results for the compositions containing ascorbic acid, threonine or serine are reproduced from Chinese patent publication CN 102138925 A.

TABLE 5
Stability at 40° C. of tigecycline in various solid compositions.

| | | Excipient | T:E | | | Storage | Total |
|---|---|---|---|---|---|---|---|
| Tigecycline | | amount | mass | pH before | Storage | time | Impurities |
| (mg) | Excipient | (mg) | ratio | lyophilization | RH (%) | (days) | (%) |
| 50 | Arginine | 82.6 | 1:1.65 | 5.1 | 75 | 0 | 0.60 |
| | | | | | | 28 | 0.71 |
| | | | | | | 56 | 0.83 |
| | | | | | | 84 | 0.99 |
| 100 | Lactose | 62 | 1:0.62 | 5.0 | 75 | 20 | 2.39 |
| | monohydrate* | 200 | 1:2 | 5.0 | | 25 | 2.73 |
| | | 200 | 1:2 | 3.0 | | 39 | 1.51 |
| | | 200 | 1:2 | 5.0 | | 48 | 3.43 |
| 50 | Ascorbic | 50 | 1:1 | 4.5 | 75 | 10 | 1.16 |
| | acid** | 25 | 1:0.5 | | | 10 | 1.09 |
| | | 50 | 1:1 | | | 90 | 2.02 |
| 50 | Threonine** | 100 | 1:2 | 3.5 | 75 | 10 | 1.20 |
| | | | | 4.5 | | 10 | 1.22 |
| | | | | 4.5 | | 90 | 1.97 |
| 50 | Serine** | 100 | 1:2 | 4.5 | 75 | 10 | 1.62 |

*Results reported in U.S. Pat. No. 7,879,828; Tables 6b, 1, 2a and 5, respectively.
**Results reported in CN 102138925 A.

The lyophilized compositions including tigecycline and arginine were more stable than comparable lyophilized compositions including tigecycline and lactose monohydrate. For example, after 28 days at 40° C., the composition containing arginine had an impurity concentration that was 74% less than the impurity concentration of the composition containing lactose monohydrate after 25 days at 40° C. The arginine composition also had an impurity concentration after 56 days at 40° C. that was 76% less than that of the lactose monohydrate composition after only 48 days at 40° C.

Although the lyophilized compositions containing ascorbic acid, threonine or serine were reported to be more stable than lyophilized compositions containing lactose monohydrate, the lyophilized compositions including tigecycline and arginine were even more stable. For example, after 28 days at 40° C., the composition containing arginine had an impurity concentration that was 35%, 41% and 56% less than the impurity concentrations of the compositions containing ascorbic acid, threonine or serine, respectively, after only 10 days at 40° C. After 84 days at 40° C., the arginine composition had an impurity concentration that was 51% and 50% less than the impurity concentrations of the ascorbic acid or threonine compositions, respectively, after 90 days at 40° C.

In the results listed in Table 5, when solid compositions containing tigecycline and arginine were stored at 40° C. over a period of 84 days, less than 2% of the tigecycline degraded, as determined by the total impurities of 0.99%. In contrast, when solid compositions containing tigecycline and lactose monohydrate were stored at 40° C. over a period of only 48 days, more than 2% of the tigecycline degraded, as determined by the total impurities of 3.43%. In addition, when solid compositions containing tigecycline and ascorbic acid were stored at 40° C. over a period of 90 days, more than 2% of the tigecycline degraded, as determined by the total impurities of 2.02%.

In the results listed in Table 5, when solid compositions containing tigecycline and arginine were stored at 40° C. over a period of 84 days, less than 1.5% of the tigecycline degraded, as determined by the total impurities of 0.99%. In contrast, when solid compositions containing tigecycline and threonine were stored at 40° C. over a period of 90 days, more than 1.5% of the tigecycline degraded, as determined by the total impurities of 1.97%.

The results of this example demonstrated that solid compositions comprising tigecycline and arginine exhibit greater stability than the stability reported for solid compositions comprising tigecycline and an excipient selected from lactose monohydrate, ascorbic acid, threonine, and serine.

### Example 4

This example demonstrates that solid compositions comprising tigecycline and arginine are more stable than solid compositions comprising tigecycline and an excipient other than arginine.

The stability of a solid composition comprising tigecycline and arginine was compared to the stability of solid compositions comprising tigecycline and one of the following lyophilization excipients: glycine, asparagine, lysine, aspartic acid, dextran, mannitol, or methylcellulose. Each composition was formed by dissolving the excipient in water and adjusting the solution to an acidic pH. Tigecycline was then added to each solution, and the solution pH was adjusted as needed to provide the pH listed in Table 6. The resulting solutions were clear, with no visible particles, and were filtered and lyophilized to form solid compositions, as described above with regard to Tables 1-3, above. Each lyophilized composition was sealed in a 5 mL vial with a 13 mm STELMI 6720GC rubber stopper, and stored at 55±2° C. (˜75% relative humidity (RH)). After 2 weeks at 55° C., a portion of the vials were visually inspected with regard to the color of the solid, and were then reconstituted with 5.3 mL of 0.9% sodium chloride injection, USP. The reconstituted liquids were visually inspected, and were then analyzed by HPLC to determine the concentrations of tigecycline and of any impurities, including the 6-ene oxidation product and the epimerization product. Table 6 lists the results of these analyses, as well as the properties of the liquids used to form the lyophilized compositions.

TABLE 6
Stability, after 2 weeks at 55 ± 2° C., of tigecycline in various solid compositions.

| | | Excipient | T:E | pH | | | |
|---|---|---|---|---|---|---|---|
| Tigecycline | | amount | mass | before | Impurities (%) | | |
| (mg/vial)¹ | Excipient | (mg/vial) | ratio | lyoph. | 6-ene | Epimer | Total |
| | Amino Acids | | | | | | |
| 53 | Arginine | 100 | 1:2 | 4.75 | 0.02 | 0.62 | 0.90 |
| 53 | Glycine | 100 | 1:2 | 4.75 | 0.10 | 7.86 | 8.17 |
| | | | | 6.8 | 0.44 | 9.85 | 11.0 |
| 53 | Asparagine | 53 | 1:1 | 4.75 | 0.24 | 4.80 | 5.38 |
| | | | | 7.8 | 0.64 | 1.21 | 2.83 |
| 53 | Lysine | 100 | 1:2 | 4.75 | 0.17 | 4.37 | 4.78 |
| | | | | 7.73 | 1.86 | 2.20 | 6.14 |
| 53 | Aspartic Acid | 100 | 1:2 | 4.75 | 0.17 | 2.55 | 3.16 |
| | Other Excipients | | | | | | |
| 53 | Dextran | 100 | 1:2 | 4.75 | 0.06 | 2.88 | 3.10 |
| 53 | Mannitol | 100 | 1:2 | 4.75 | 0.07 | 5.80 | 6.10 |
| 53 | Methylcellulose | 53 | 1:2 | 4.75 | 0.18 | 5.20 | 5.72 |

¹Amount of tigecycline includes 6% overage.

The lyophilized composition including tigecycline and arginine was more stable than comparable lyophilized compositions including tigecycline and other amino acids. After 2 weeks at 55° C., the composition containing arginine had a total impurity amount that was 89% less than that of the composition containing glycine having a pH of 4.75 before lyophilization, 68% less than that of the composition containing asparagine having a pH of 7.8 before lyophilization, 85% less than that of the composition containing lysine having a pH of 7.73 before lyophilization, and 72% less than that of the composition containing aspartic acid.

The lyophilized composition including tigecycline and arginine was more stable than comparable lyophilized compositions including tigecycline and other lyophilization excipients. After 2 weeks at 55° C., the composition containing arginine had an impurity concentration that was 71% less than that of the composition containing dextran, 85% less than that of the composition containing mannitol, and 84% less than that of the composition containing methylcellulose.

In the results listed in Table 6, when solid compositions containing tigecycline and arginine were stored at 55° C. over a period of 2 weeks, less than 2% of the tigecycline degraded, as determined by the total impurities of 0.90%. In contrast, when solid compositions containing tigecycline in combination with arginine, glycine, asparagine, lysine, aspartic acid, dextran, mannitol or methylcellulose were stored at 55° C. over a period of 2 weeks, more than 2% of the tigecycline degraded, as determined by the total impurities of 2.83% for the asparagine composition, and by the higher impurity levels for the other compositions.

The results of this example demonstrated that solid compositions comprising tigecycline and arginine exhibit greater stability than solid compositions comprising tigecycline and an excipient selected from glycine, asparagine, lysine, aspartic acid, dextran, mannitol, and methylcellulose.

### Example 5

This example demonstrates the stability of solid compositions comprising tigecycline and arginine prepared under varying lyophilization conditions.

The stability of tigecycline in lyophilized compositions containing arginine was determined in compositions having a range of arginine concentrations, formed from liquids having a range of pH values, and using two different primary drying temperatures during lyophilization. Table 7 lists the results of stability analyses of lyophilized compositions containing tigecycline and arginine, where the arginine was present at one of a variety of concentrations from 50 mg/vial to 100 mg/vial, the pH was from 4.5 to 5.75, and the primary drying during lyophilization was −25° C. or −30° C. The compositions were formed and sealed in 5 mL vials as described with regard to Tables 1-3, above. Each lyophilized composition was stored for 2 weeks at 55±2° C. (˜75% RH) and then analyzed for impurities as described with regard to Tables 1-3, above. Table 7 lists the results of these analyses, as well as the concentrations of arginine in the lyophilized compositions.

TABLE 7
Stability, after 2 weeks at 55 ± 2° C., of tigecycline in solid
compositions with various concentrations of arginine.

| | | | Primary | | | |
|---|---|---|---|---|---|---|
| Tigecycline | Arginine | pH before | dry temp. | Impurities (%) | | |
| (mg/vial)¹ | (mg/vial)¹ | lyophilization | (° C.) | 6-ene | Epimer | Total |
| 53 | 50 | 4.5 | −25 | 0.03 | 1.19 | 1.52 |
| | | | −30 | 0.02 | 0.35 | 0.99 |
| | | 5.1 | −25 | 0.03 | 1.00 | 1.41 |
| | | | −30 | 0.03 | 1.00 | 1.42 |
| | | 5.75 | −25 | 0.04 | 0.90 | 1.58 |
| | | | −30 | 0.03 | 0.88 | 1.45 |
| 53 | 62.5 | 4.5 | −25 | 0.03 | 0.98 | 1.28 |
| | | | −30 | 0.03 | 0.93 | 1.25 |
| | | 5.1 | −25 | 0.03 | 0.79 | 1.15 |
| | | | −30 | 0.03 | 0.78 | 1.17 |
| | | 5.75 | −25 | 0.04 | 0.70 | 1.30 |
| | | | −30 | 0.03 | 0.70 | 1.19 |
| 53 | 75 | 4.5 | −25 | 0.03 | 0.83 | 1.14 |
| | | | −30 | 0.03 | 0.83 | 1.14 |
| | | 5.1 | −25 | 0.03 | 0.67 | 1.04 |
| | | | −30 | 0.03 | 0.65 | 1.01 |
| | | 5.75 | −25 | 0.04 | 0.59 | 1.14 |
| | | | −30 | 0.03 | 0.56 | 1.02 |
| 53 | 87.5 | 4.5 | −25 | 0.03 | 0.74 | 0.99 |
| | | | −30 | 0.03 | 0.69 | 1.00 |
| | | 5.1 | −25 | 0.03 | 0.59 | 0.93 |
| | | | −30 | 0.03 | 0.54 | 0.88 |
| | | 5.75 | −25 | 0.03 | 0.51 | 0.90 |
| | | | −30 | 0.03 | 0.49 | 0.91 |
| 53 | 100 | 4.5 | −25 | 0.02 | 0.68 | 0.95 |
| | | | −30 | 0.03 | 0.60 | 0.91 |
| | | 5.1 | −25 | 0.03 | 0.51 | 0.84 |
| | | | −30 | 0.03 | 1.16 | 1.51 |
| | | 5.75 | −25 | 0.03 | 0.47 | 0.87 |
| | | | −30 | 0.03 | 0.43 | 0.82 |

¹Amounts of tigecycline and arginine include 6% overage.

Tigecycline was stabilized in the lyophilized compositions, regardless of the concentration of arginine in the compositions, the pH of the lyophilization liquid, or the primary drying temperature, within the listed ranges. The highest amount of impurity was 1.58%, measured for a composition having 50 mg/vial arginine and formed from a lyophilization liquid having a pH of 5.75 using a primary drying temperature of −25° C. The lowest amount of impurity was 0.82%, measured for a composition having 100 mg/vial arginine and formed from a lyophilization liquid having a pH of 5.75 using a primary drying temperature of −30° C. Thus, the combination of tigecycline with arginine appeared to stabilize the tigecycline in a variety of different solid compositions. Specifically, solid compositions having a mass ratio of tigecycline to arginine of from 1:1 to 1:2, and formed from liquids having a pH before lyophilization of from 4.5 to 5.75, allowed for less than 1.6% impurities in the composition when stored for 2 weeks at 55±2° C.

The results of this example demonstrated that tigecycline was stabilized in the lyophilized compositions over wide ranges in concentration of arginine in the compositions, pH of the lyophilization liquid, and primary drying temperature.

### Example 6

This example demonstrates that solid compositions comprising tigecycline and arginine exhibit greater stability when compared with the TYGACIL™ formulation.

Table 8 provides a summary of the amount of tigecycline epimer, 6-ene oxidation product, and total impurities (imp) formed in three lots of a composition according to the invention as compared to a lot of the TYGACIL™ formulation following storage at 40° C. Each vial of each lot of the composition of the invention contained 53 mg tigecycline and 87.5 mg arginine, and demonstrated a reconstituted pH in the range of 4.25-5.75. Each vial of the TYGACIL™ formulation contained 53 mg tigecycline and 106 mg lactose monohydrate, and demonstrated a reconstituted pH of approximately 5.

TABLE 8
Stability at 40 ± 2° C./75 ± 5% RH of tigecycline in solid compositions
containing arginine or lactose monohydrate.

| | | Impurity (%) | | | | |
|---|---|---|---|---|---|---|
| Composition | | Initial | 1-month | 2-month | 3-month | 6-month |
| Lot 1 | epimer | 0.22 | 0.39 | 0.44 | 0.46 | 0.64 |
| (invention) | 6-ene | <0.05 | <0.05 | <0.05 | <0.05 | 0.07 |
| | total imp | 0.33 | 0.46 | 0.56 | 0.59 | 0.78 |
| Lot 2 | epimer | 0.22 | 0.39 | 0.46 | 0.50 | 0.70 |
| (invention) | 6-ene | <0.05 | 0.05 | 0.05 | <0.05 | 0.10 |
| | total imp | 0.35 | 0.50 | 0.64 | 0.67 | 1.0 |
| Lot 3 | epimer | 0.15 | 0.36 | 0.49 | 0.48 | 0.77 |
| (invention) | 6-ene | <0.05 | 0.06 | 0.05 | <0.05 | 0.09 |
| | total imp | 0.28 | 0.49 | 0.67 | 0.65 | 1.1 |
| TYGACIL ™ | epimer | 0.66 | 1.09 | 1.25 | 1.44 | NT |
| | 6-ene | <0.05 | 0.06 | <0.05 | <0.05 | NT |
| | total imp | 0.79 | 1.26 | 1.31 | 1.50 | NT |

NT: not tested

Solid compositions comprising tigecycline and arginine according to the invention also demonstrate excellent stability at 25° C. Table 9 provides a summary of the amount of tigecycline epimer, 6-ene oxidation product, and total impurities (imp) formed in three lots of a composition according to the invention following storage at 25° C. Each vial of each lot of the composition of the invention contained 53 mg tigecycline and 87.5 mg arginine, and demonstrated a reconstituted pH in the range of 4.25-5.75.

TABLE 9
Stability at 25 ± 2° C./60 ± 5% RH of tigecycline in solid compositions containing arginine

| | | Impurity (%) | | | | | | |
|---|---|---|---|---|---|---|---|---|
| Lot # | | Initial | 1-mo | 2-mo | 3-mo | 6-mo | 9-mo | 12-mo |
| 1 | epimer | 0.22 | 0.27 | 0.29 | 0.27 | 0.33 | 0.35 | 0.36 |
| | 6-ene | <0.05 | <0.05 | <0.05 | <0.05 | 0.05 | <0.05 | <0.05 |
| | total | 0.33 | 0.33 | 0.38 | 0.36 | 0.41 | 0.45 | 0.51 |
| 2 | epimer | 0.22 | 0.27 | 0.26 | 0.27 | 0.36 | 0.34 | 0.41 |
| | 6-ene | <0.05 | 0.05 | <0.05 | <0.05 | 0.10 | 0.05 | 0.06 |
| | total | 0.35 | 0.39 | 0.41 | 0.42 | 0.61 | 0.56 | 0.59 |
| 3 | epimer | 0.15 | 0.27 | 0.32 | 0.25 | 0.39 | 0.34 | 0.36 |
| | 6-ene | <0.05 | 0.07 | <0.05 | <0.05 | 0.10 | 0.06 | 0.08 |
| | total | 0.28 | 0.43 | 0.50 | 0.38 | 0.60 | 0.56 | 0.52 |

The results of this example demonstrate that solid compositions comprising tigecycline and arginine exhibit greater storage stability the TYGACIL™ formulation comprising tigecycline and lactose monohydrate.

### Example 7

This example compares the storage stability of solutions formed from solid compositions comprising tigecycline and arginine to solutions formed from solid compositions comprising tigecycline and lactose monohydrate.

Solid lyophilized compositions containing 53 mg tigecycline and either 87.5 mg arginine or 100 mg lactose monohydrate were prepared. Each solid was reconstituted in a 5.3 mL volume of 0.9% sodium chloride reconstitution liquid, to provide reconstituted solutions having tigecycline concentrations of 10 mg/mL. Aliquots of 5 mL of each reconstituted solution, which contained 50 mg tigecycline, were then diluted with 50 mL of an infusion liquid to provide diluted solutions having final concentrations of 1 mg/mL. Each diluted solution was stored either at 5° C. for 48 hours, or at 25° C. for 24 hours. After storage, the liquids were analyzed by HPLC to determine the concentrations of tigecycline and of any impurities, including the 6-ene oxidation product and the epimerization product. Table 10 lists the results of these analyses, as well as the properties of the diluted solutions.

TABLE 10
Stability of tigecycline in reconstituted and diluted liquids.

| | | Excipient | Storage | Storage | pH of | | | |
|---|---|---|---|---|---|---|---|---|
| Infusion | | amount | temp | time | diluted | Impurities (%) | | |
| Liquid | Excipient | (mg) | (° C.) | (hours) | liquid | 6-ene | Epimer | Total |
| 0.9% | Arginine | 82.6 | 25 | 0 | 5.02 | 0.08 | 0.73 | 0.92 |
| NaCl | | | | 24 | 5.02 | 0.09 | 1.12 | 1.42 |
| injection | | | 5 | 0 | 5.00 | 0.05 | 0.37 | 0.50 |
| | | | | 48 | 5.06 | 0.09 | 0.46 | 0.63 |
| | Lactose | 100 | 25 | 0 | 5.07 | 0.10 | 1.19 | 1.47 |
| | monohydrate | | | 24 | 5.05 | 0.09 | 1.59 | 1.90 |
| | | | 5 | 0 | 5.00 | 0.06 | 0.91 | 1.13 |
| | | | | 48 | 5.05 | 0.08 | 0.99 | 1.24 |
| 5% | Arginine | 82.6 | 25 | 0 | 4.74 | 0.08 | 0.83 | 1.02 |
| Dextrose | | | | 24 | 4.72 | 0.08 | 1.50 | 1.74 |
| injection | | | 5 | 0 | 4.70 | 0.06 | 0.39 | 0.55 |
| | | | | 48 | 4.71 | 0.09 | 0.52 | 0.71 |
| | Lactose | 100 | 25 | 0 | 4.70 | 0.09 | 1.26 | 1.54 |
| | monohydrate | | | 24 | 4.69 | 0.07 | 1.93 | 2.18 |
| | | | 5 | 0 | 4.69 | 0.06 | 0.93 | 1.20 |
| | | | | 48 | 4.71 | 0.07 | 1.07 | 1.30 |
| Lactated | Arginine | 82.6 | 25 | 0 | 5.28 | 0.08 | 1.10 | 1.30 |
| Ringer's | | | | 24 | 5.27 | 0.09 | 1.85 | 2.21 |
| injection | Lactose | 100 | 25 | 0 | 5.31 | 0.09 | 1.55 | 1.81 |
| | monohydrate | | | 24 | 5.31 | 0.07 | 2.27 | 2.59 |

Tigecycline was more stable in reconstituted and diluted liquids formed from solid compositions containing arginine than in those formed from conventional solid compositions containing lactose monohydrate. For example, when 0.9% sodium chloride injection was used as the infusion liquid, the diluted liquid formed from a solid composition containing arginine had an initial impurity level of 0.92% at 25° C., whereas the diluted liquid formed from a solid composition containing lactose monohydrate had a higher initial impurity level of 1.47% at 25° C. After storage at 25° C. for 24 hours, the impurity levels of the liquids were 1.42% and 1.90%, respectively. Similar room temperature stability trends were observed when the infusion liquid was 5% dextrose injection or lactated Ringer's injection.

The increased stability associated with arginine also was observed during refrigerated storage. For example, when 0.9% sodium chloride injection was used as the infusion liquid, the diluted liquid formed from a solid composition containing arginine had an initial impurity level of 0.50% at 5° C., whereas the diluted liquid formed from a solid composition containing lactose monohydrate had a higher initial impurity level of 1.13% at 5° C. After storage at 5° C. for 48 hours, the impurity levels of the liquids were 0.63% and 1.24%, respectively. Similar low temperature stability trends were observed when the infusion liquid was 5% dextrose injection.

The results of this example demonstrated that solutions formed from solid compositions comprising tigecycline and arginine exhibit equivalent or greater stability than solutions formed from solid compositions comprising tigecycline and lactose monohydrate.

All references, including publications, patent applications, and patents, cited herein are hereby incorporated by reference to the same extent as if each reference were individually and specifically indicated to be incorporated by reference and were set forth in its entirety herein.

The use of the terms “a” and an and the and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A composition, comprising:
tigecycline, and
arginine;
where the composition is a solid.

2. The composition of claim 1, comprising from 10 to 200 mg tigecycline.

3. The composition of claim 2, comprising from 10 to 750 mg of arginine.

4. The composition of claim 1, where the mass ratio of tigecycline to arginine is from 1:0.5 to 1:5.

5. The composition of claim 1, where
when the composition is combined with an aqueous carrier liquid to form a solution, the solution has a pH of from 4 to 8.

6. The composition of claim 1, where the solution has a pH of from 4.75 to 5.25.

7. The composition of claim 1, where when the composition is stored at 40° C. over a period of 6 months, at most 2% of the tigecycline degrades.

8. The composition of claim 1, where when the composition is stored at 25° C. over a period of 12 months, at most 2.4% of the tigecycline degrades.

9. The composition of claim 1, where when the composition is combined with an aqueous carrier liquid to form a solution to form a solution having a tigecycline concentration of 1 mg/mL and then stored at 25° C. for 24 hours, at most 1.5% of the tigecycline degrades.

10. The composition of claim 1, where when the composition is combined with an aqueous carrier liquid to form a solution having a tigecycline concentration of 1 mg/mL and then stored at 5° C. for 48 hours, at most 1% of the tigecycline degrades.

11. A composition, comprising:
50 to 60 mg tigecycline, and
arginine;
where the mass ratio of arginine to tigecycline is from 1:0.5 to 1:5,
the composition is a solid, and
when the composition is combined with 5.3 mL of an aqueous carrier liquid to form a solution, the solution has a pH of from 4 to 8.

12. The composition of claim 11, where the mass ratio of arginine to tigecycline is from 1:1 to 1:2.

13. The composition of claim 11, where the solution has a pH of from 4.25 to 5.75.

14. The composition of claim 11, where when the composition is stored at 40° C. over a period of 6 months, at most 1% of the tigecycline degrades.

15. The composition of claim 11, where when the composition is stored at 25° C. over a period of 12 months, at most 2.4% of the tigecycline degrades.

16. The composition of claim 11, where when the solution is stored at 5° C. for 48 hours, at most 1% of the tigecycline degrades.

17. A method of stabilizing a tigecycline composition, the method comprising:
forming a liquid mixture comprising
a solvent,
tigecycline, and
arginine; and
lyophilizing the liquid mixture,
thereby stabilizing a tigecycline composition.

18. The method of claim 17, where the mass ratio of tigecycline to arginine is from 1:0.5 to 1:5.

19. The method of claim 17, where when the composition is stored at 25° C. over a period of 12 months, at most 2.4% of the tigecycline degrades.

20. A method of treating a bacterial infection in a subject, the method comprising (i) combining the composition of claim 1 with an aqueous carrier liquid to form a solution comprising an effective amount of tigecycline, and (ii) administering the solution to the subject, thereby treating the bacterial infection in the subject.
